(19) United States
(12) Patent Application Publication
(10) Pub. No.: US 2006/0134049 A1
(43) Pub. Date: Jun. 22, 2006

(54) Hair styling compositions

(76) Inventors: Andrea Claudette Keenan, Plymouth Meeting, PA (US); Charles Elwood Jones, Yardley, PA (US); Miao Wang, Horsham, PA (US)

Correspondence Address: ROHM AND HAAS COMPANY;PATENT DEPARTMENT, 100 INDEPENDENCE MALL WEST, PHILADELPHIA, PA, 19106-2399, US

(21) Appl. No.: 11/015,972

(22) Filed: Dec. 17, 2004

(51) Int. Cl. A61K 8/81

(52) U.S. Cl. 424/70.15; 424/70.16

(57) ABSTRACT

The present invention relates to compositions and methods for treating and modifying hair. More particularly, this invention is directed to styling and conditioning compositions for modifying and fixing hair comprising one or more rheology modifiers and one or more hair styling resins and a method of using the compositions to modify, condition and fix hair.

The present invention relates to compositions and methods for treating and modifying hair. More particularly, this invention is directed to styling and conditioning compositions for modifying and fixing hair comprising one or more rheology modifiers and one or more hair styling resins and a method of using the compositions to modify, condition and fix hair.

Hair tends to return and recover to its initial shape or position after it is chemically and or physically modified so, as an example, it does not hold a set well. Hair styling and fixative products help create and establish chemically interactive forces between hair fibers, including those which provide, as an example, adherence to the adjacent hairs so they can attain and maintain a particular shape or configuration as the polymer is applied, dries, and remains in contact with hair over time. In the past, hairsprays have dominated the styling aid market because of easy use, good styling characteristics, and simple application. Pump hair sprays, hydrocarbon and dimethylether containing aerosols, and carbon dioxide aerosols are three major types of sprays. However, non-aerosol and water-based styling aid products such as styling gels, glazes, spray foams, styling creams and waxes, and styling lotions have been gradually replacing hairsprays, partially due to the shift in the market away from products which contain volatile organic compounds.

A hair styling gel is a firm gel that thins upon application of shear such that it spreads thinly when applied to hair. Hair styling glazes are easy to spread, clear flowable gels that are particularly useful for the wet look or blow dry styling methods. Hair styling creams are easy to spread, flowable lotions. Such hair styling products typically comprise a hair fixative resin and a variety of adjuvants. The most significant of these adjuvants include rheology modifiers which are intended to thicken the composition into the form of a gel. Many rheology modifiers when combined with hair fixative resins result in a composition which gives a stiff, crunchy feel to the hair, but unfortunately, they often have poor resistance to humidity, or they tend to flake on the hair, or both. To compensate for the flaking, formulators will often add plasticizers to the composition. However, this often results in lower stiffness. One of the most widely used hair fixative resins for hair gels is polyvinylpyrrolidinone (“PVP”). The most common rheology modifier used in combination with PVP is known under its INCI name of Carbomer.

We have discovered that combinations of certain rheology modifiers with certain hair fixative resins results in compositions which, when applied to the hair, provide improved stiffness over that expected of the hair fixative resin when it is used with typical rheology modifiers like Carbomer, and which does not suffer from poor resistance to humidity. Such compositions comprise:
a) one or more rheology modifiers selected from the group consisting of non-crosslinked hydrophobically modified poly(meth)acrylates, amino modified acrylates, acrylonitrogen modified (meth)acrylates, hydrophobically modified polyethers, and combinations thereof, and
b) one or more hair fixative resins selected from the group consisting of: acrylates/hydroxyesters acrylates copolymer, AMP-acrylates/allyl methacrylate copolymer, PVM/MA copolymer, butyl ester of PVM/MA copolymers, ethyl ester of polyvinyl methyl ether/methacrylate copolymers, ethyl ester of PVM/MA copolymer, guar hydroxypropyl trimonium chloride, guar hydroxypropyl trimonium chloride, isobutylene/ethylmaleimide/hydroxyethylmaleimide copolymers, modified corn starch, octylacrylamide/acrylates butylaminoethyl methacrylate copolymer, octylacrylamide/acrylates butylaminoethyl methacrylate copolymers, poly(methacrylic acid/acrylamidomethyl propane sulfonic acid), Polyimide-1, polyquaternium-10, polyquaternium-11, polyquaternium-16, polyquaternium-2, polyquaternium-28, polyquaternium-28 and dimethicones, polyquaternium-39, polyquaternium-4, polyquaternium-46, polyquaternium-55, polyquaternium-7, polyvinylcaprolactam acrylate copolymers, polyvinylpyrrolidinone (“PVP”), PVP and dimethicones, PVP/dimethylaminoethyl methacrylate copolymers, PVP/dimethylaminoethylmethacrylate copolymer, PVP/DMAPA acrylate copolymers, PVP/DMAPA acrylates copolymer, PVP/vinyl acetate copolymer, PVP/vinylcaprolactam/DMAPA acrylate copolymers, VA/Crotonate copolymer, VA/Crotonates/vinyl neodecanoate copolymer, vinyl acetate/crotonates/vinyl neodecanoate copolymers, vinyl acetate/crotonic acid copolymer, vinyl acetate/crotonic acid copolymers, vinyl caprolactam/PVP/dimethyl aminoethyl methacrylate copolymer, vinyl caprolactam/PVP/dimethyl aminoethyl methacrylate copolymers, VP/acrylates/lauryl methacrylate copolymer, VP/methacrylamide/vinyl imidazole copolymer, polymers of acrylamide/acrylamidomethylpropanesulfonate/methacrylic acid, and combinations thereof.

Such compositions optionally further comprise one or more additives such as, for example: additional thickeners, additional rheology modifiers, additional hair fixative resins, other polymers, neutralizers, humectants, surfactants, conditioning agents, silicones, colors, dyes, fragrances, naturally occurring materials, and preservatives.

Unless otherwise specified, the following abbreviations are used in describing the polymers used in the composition of the invention:
AMP: aminomethylpropanol
DMAPA dimethylaminopropylamine
PVM/MA polyvinylmethyl ether/maleic anhydride
PEG polyethylene glycol
PQ polyquaternium
PVP polyvinylpyrrolidinone
VA vinyl acetate
VP vinyl pyrrolidone

Unless otherwise specified, polymer names are taken from the Internatitonal Cosmetic Ingredient Dictionary and Handbook, 9^{th} ed., 2002, Pub. by the Cosmetic, Toiletry, and Fragrance Association, Washington, D.C. (“INCI”)

Another embodiment of this invention is a process for treating and modifying hair comprising the steps of applying to hair a polymer composition comprising:
a) one or more rheology modifiers selected from the group consisting of non-crosslinked hydrophobically modified poly(meth)acrylates, amino modified acrylates, acrylonitrogen modified (meth)acrylates, hydrophobically modified polyethers, and combinations thereof,
b) one or more hair fixative resins selected from the group consisting of: acrylates/hydroxyesters acrylates copolymer, AMP-acrylates/allyl methacrylate copolymer, PVM/MA copolymer, butyl ester of PVM/MA copolymers, ethyl ester of polyvinyl methyl ether/methacrylate copolymers, ethyl ester of PVM/MA copolymer, guar hydroxypropyl trimonium chloride, guar hydroxypropyl trimonium chloride, isobutylene/ethylmaleimide/hydroxyethylmaleimide copolymers, modified corn starch, octylacrylamide/acrylates butylaminoethyl methacrylate copolymer, octylacrylamide/acrylates butylaminoethyl methacrylate copolymers, poly(methacrylic acid/acrylamidomethyl propane sulfonic acid), polymers of acrylamide/acrylamidomethylpropanesulfonate/methacrylic acid, Polyimide-1, polyquaternium-10, polyquaternium-11, polyquaternium-16, polyquaternium-2, polyquaternium-28, polyquaternium-28 and dimethicones, polyquaternium-39, polyquaternium-4, polyquaternium-46, polyquaternium-55, polyquaternium-7, polyvinylcaprolactam acrylate copolymers, polyvinylpyrrolidinone (“PVP”), PVP and dimethicones, PVP/dimethylaminoethyl methacrylate copolymers, PVP/dimethylaminoethylmethacrylate copolymer, PVP/DMAPA acrylate copolymers, PVP/DMAPA acrylates copolymer, PVP/vinyl acetate copolymer, PVP/vinylcaprolactam/DMAPA acrylate copolymers, VA/Crotonate copolymer, VA/Crotonates/vinyl neodecanoate copolymer, vinyl acetate/crotonates/vinyl neodecanoate copolymers, vinyl acetate/crotonic acid copolmer, vinyl acetate/crotonic acid copolymers, vinyl caprolactam/PVP/dimethyl aminoethyl methacrylate copolymer, vinyl caprolactam/PVP/dimethyl aminoethyl methacrylate copolymers, VP/acrylates/lauryl methacrylate copolymer, VP/methacrylamide/vinyl imidazole copolymer, and combinations thereof; and, optionally, c) one or more additives.

For purposes of this invention, “hydrophobically modified poly(meth)acrylate” means acrylates/C10-30 alkyl acrylate crosspolymer, acrylates/C12-22 alkylmethacrylate copolymer, acrylates/steareth-20 methacrylate copolymer, acrylates/steareth (or ceteth)-20 itaconate copolymer, acrylates/steareth-50 acrylate copolymer, steareth-10 allyl ether/acrylate copolymer, acrylates beheneth-25 methacrylate copolymer, acrylates/vinyl acetate crosspolymer, glyceryl polymethacrylate, and ammonium acryloyldimethyltaurate/beheneth-25 methacrylate copolymer; “amino modified acrylates” means acrylates/aminoacrylates/C10-30 alkyl PEG-20 itaconate copolymer; “hydrophobically modified polyethers” means hydrophobically modified ethoxylated urethanes such as, for example, PEG-150/stearyl alcohol/SMDI copolymer, and hydrophobic polyethers endcapped by fatty acid esters such as, for example, PEG-150 distearate, PEG-55 propylene glycol oleate, and PEG-150 pentaerythrityl tetrastearate.

In a separate embodiment of this invention, we have discovered a hair styling composition in which the rheology modifier and the hair fixative resin are the same. That is, the rheology modifier is also acting as the hair fixative resin. The preferred rheology modifier for such compositions is acrylates beheneth-25 methacrylate copolymer, which is sold under the tradename Aculyn™ 28 rheology modifier (Rohm and Haas Company, Philadelphia, Pa., USA).

In one embodiment of this invention, the rheology modifier is selected from one or more of hydrophobically modified (meth)acrylates such as, for example, acrylates beheneth-25 methacrylate copolymer and acrylates/steareth-20 methacrylate copolymers. In another embodiment of this invention, the hair styling composition further comprises one or more additives selected from non-associative (meth)acrylate thickeners such as, for example, acrylates copolymer and acrylates crosspolymer.

As used herein, the term “hydrophobic” refers to monoethylenically unsaturated monomers and polymers which have low water solubility under the conditions of emulsion polymerization, as described in U.S. Pat. No. 5,521,266. The term “copolymer” refers to polymer compositions containing units of two or more different monomers, the term “terpolymer” refers to polymer compositions containing units of three or more different monomers, and the term “tetrapolymer” refers to polymer compositions containing units of four or more different monomers.

As used herein, the following terms have the designated definitions, unless the context clearly indicates otherwise. The term “alkyl (meth)acrylate” refers to either the corresponding acrylate or methacrylate ester; similarly, the term “(meth)acrylic” refers to either acrylic or methacrylic acid and the corresponding derivatives, such as esters or amides.

All percentages referred to will be expressed in weight percent (%), based on total weight of polymer or composition involved, unless specified otherwise. The following abbreviations are used herein: g=grams; ppm=parts per million by weight/volume. Unless otherwise specified, ranges listed are to be read as inclusive and combinable and temperatures are in degrees centigrade (° C.).

The rheology modifiers of the present invention typically have a weight average molecular weight (M_{W}) for the backbone polymer of 200 to 1,200,000 preferably from 2,000 to 1,000,000 and more preferably from 200,000 to 1,000,000. Weight average molecular weights for the backbone polymer are based on aqueous phase gel permeation chromatography (GPC) analysis using known polymer standards appropriate for the compositions involved. The rheology modifiers are subjected to hydrolysis (to the acid form) prior for determination of the backbone polymer molecular weight.

The rheology modifiers of this invention may be prepared by conventional polymerization methods well known to those skilled in the art including, for example, emulsion, solution, bulk, and suspension polymerization. The (meth)acrylic polymers are preferably prepared by emulsion polymerization such as those methods disclosed in U.S. Pat. Nos. 3,245,932, 3,453,245 and 4,196,190.

The compositions of this invention are used as compositions for treating hair by incorporating them in a cosmetically acceptable medium in amounts of from 0.1 to 10 weight percent, preferably from 0.5 to 5 weight percent, based on total polymer solids.

The rheology modifiers and hair fixative resins of the invention can be incorporated as compositions and formulations in various forms such as, for example, hair spray, styling gel, styling glaze, spray foam, styling cream, styling wax, styling lotion, liquid foam, and mousse. They may contain water and also any cosmetically acceptable solvent such as, for example, monoalcohols, such as alkanols having 1 to 8 carbon atoms such as, for example, ethanol, isopropanol, benzyl alcohol, and phenylethyl alcohol; polyalcohols such as, for example, alkylene glycols such as, for example, glycerins, ethylene glycol, and propylene glycol; and glycol ethers such as, for example, mono-, di- and tri-ethylene glycol monoalkyl ethers such as, for example, ethylene glycol monomethyl ether, ethylene glycol monoethyl ether, and diethylene glycol monomethyl ether, used singly or in a mixture. These solvents can be present in proportions of up to as much as 99.5 percent by weight, relative to the weight of the total composition.

The compositions of this invention may include one or more additives such as, for example, thickeners, additional rheology modifiers, other hair fixative polymers, other polymers, neutralizers, humectants, surfactants, conditioning agents, silicones, colors, dyes, fragrances, naturally occurring materials and preservatives; and can be neutralized at any stage of preparing the formulation. Suitable naturally occurring materials include soy bean oil, cellulose, modified cellulose, castor oil and linseed oil. In addition, the compositions of this invention also can contain any other ingredient normally used in cosmetics such as, for example, perfumes, dyestuffs (also referred to as coloring agents and colorants) which can serve to color the composition itself or the fibers (also referred to as follicles) of the hair, preservatives, sequestering agents, thickeners, silicones, softeners, foam synergistic agents, foam stabilizers, sun filters, peptizing agents and also anionic, non-ionic, cationic or amphoteric surface-active agents or mixtures thereof.

Additional thickeners or rheology modifiers which can be used in the compositions of this invention include, for example, non-associative (meth)acrylate thickeners such as, for example, partially crosslinked anionic poly (meth)acrylic acid polymers such as, for example, polyacrylic acid cross-linked with allyl ethers of pentaerythrol or allyl ethers of sucrose and commonly referred to as “Carbomer” (available from Noveon, under the tradename Carbopol™), and their many variants, polyacrylates crosslinked with other acrylate type monomers that fall within the INCI classification of acrylates copolymer or acrylates crosspolymer, sodium polyacrylate or sodium acrylates copolymer; acrylates/C10-30 alkylacrylates crosspolymer, paraffinum liquidum, and PPG-1 trideceth-6, sold under the SalCare™ SC91 tradename (Ciba Specialty Chemicals Corporation, High Point, N.C.); other non-associative thickeners such as, for example, polyacrylamide, acrylamide/ammonium acrylate copolymer, acrylates/acrylamide copolymer, poly ethylene oxide, copolymers of ethylene oxide and propylene oxide, polyvinyl alcohol, poly vinyl pyrrolidone, optionally crosslinked, acrylates/acrylonitrogen copolymer, ammonium acryloyldimethyltaurate/vinyl pyrrolidinone copolymer, and methylmethacrylate crosspolymer; and thickeners or rheology modifiers derived from oleochemicals, that is, with a “natural” backbone such as, for example, alginates such as, for example, algin, propylene glycol alginate; carrageenan (kappa, iota, lambda versions); gums such as, for example, xanthan gum, sclerotium gum, tragacanth gum; celluluse gums; chitosan and chitosan lactate; hydroxyalkylcellulose such as, for example, methylcellulose, methylhydroxyethyl cellulose, cetyl hydroxyethyl cellulose, ethylcellulose, hydroxyethylcellulose, hydroxypropylcellulose, hydroxypropylmethylcellulose, cetylhydroxycellulose, hydroxymethylcellulose, methyl hydroxyethylcellulose, hydroxypropylstarch phosphate, guar, and hydroxypropylguar; modified celluloses such as, for example, PEG-120 methyl glucose dioleate; starch, and modified starches such as, for example, hydroxypropyl starch phosphate; gelatin; gum arabic/gum acacia, gum karaya; and maltodextrin and pectin; and miscellaneous thickeners such as, for example, Polyvinyl methyl ether/methacrylate decadiene crosspolymer and trihydroxystearin.

Compositions of the invention are applied to wet or dry hair by spraying or by rubbing onto the hair manually. The treated hair is then mechanically fixed in the desired configuration. In the case of application to wet hair, the hair may then dried using ambient air or electric or hot air drying. The hair may then combed to provide the desired hairstyle. After use, the hair may be rinsed with water to remove the hair fixative.

Other optional ingredients are also incorporated into the hair styling compositions of this invention. The identity of the optional ingredients is not limited as long as the optional ingredients do not adversely affect the aesthetics or efficacy of the hair styling composition. Such optional ingredients are well known to those skilled in the art and include emulsifiers such as, for example, anionic or nonionic surfactants; preservatives such as, for example, benzyl alcohol, methyl paraben, propyl paraben, or imidazolidinylurea; cationic conditioners such as cetyl trimethyl ammonium chloride, methyldibromoglutaronitrile (available from ONDEO Nalco, Naperville, Ill. under the tradename Merguard™), stearyl dimethyl benzyl ammonium chloride, isothiazolones such as, for example, Kathon™ and Neolone™ (available from Rohm and Haas Company, Philadelphia, Pa.), and di(partially hydrogenated tallow) dimethyl ammonium chloride; coloring agents such as, for example, any of the FD&C or D&C dyes; perfume oils; and chelating agents such as, for example, ethylenediaminetetraacetic acid

Hair styling compositions of this invention may also contain other conventional hair care adjuvants such as, for example, plasticizers such as, for example, glycols, phthalate esters and glycerine; silicones; emollients; lubricants; and penetrating agents such as, for example, various lanolin compounds, protein hydrolysates and other protein derivatives, ethylene adducts, and polyoxyethylene cholesterol, fragrances, and fragrance solubilizers.

The compositions of this invention may also contain electrolytes, such as, for example, aluminum chlorohydrate, alkali metal salts, e.g., sodium, potassium or lithium salts, these salts preferably being halides, such as the chloride or bromide, and the sulphate, or salts with organic acids, such as the acetates or lactates, and also alkaline earth metal salts, preferably the carbonates, silicates, nitrates, acetates, gluconates, pantothenates and lactates of calcium, magnesium and strontium. Other suitable electrolytes include, for example, metal cross-linking agents such as, for example, salts of magnesium, calcium, and zinc.

Hair styling compositions of the present invention are typically prepared by dissolving the components in water or a water/alcohol mixture, with heating if necessary and any optional ingredients are then added and the mixture is stirred to provide the composition.

When the hair styling is in the form of a hair spray or mousse, it may additionally contains up to 50 weight percent of one or more propellants. Typical propellants include, for example, ethers, compressed gases, halogenated hydrocarbons and hydrocarbons, dimethyl ether, carbon dioxide, nitrogen, nitrous oxide and volatile hydrocarbons, such as butane, isobutane, and propane.

The hair styling composition of the invention may also be incorporated into other useful compositions and formulations such as, for example, gels, setting agents, setting creams, pomade, waxing agents, oil treatments, foams, mousses, gels that can be sprayed, shine agents, conditioners left on skin and hair, conditioning agents, softeners, rinse off conditioners, shampoos, shampoos including conditioners, hair color treatments, hair bleaching treatments, agents for increasing hair volume, moisturizers, soaps, cosmetics, body washes, shaving preparations such as, for example, lotions, creams, gels and glazes, sunscreens, and topical skin and eye treatments.

The hair styling compositions can also be formulated with other ingredients known to the cosmetic industry and registered under CTFA International Cosmetic Ingredients Dictionary and Handbook. Such ingredients include emollients, humectants, film forming polymers, propellants, solvents, silicones, pigments, dyes, buffers, organic and inorganic suspending and thickening agents, waxes, surfactants and co-surfactants, plasticizers, organic and inorganic neutralizing agent, preservatives, flavoring agents, perfumes, and active ingredients including sunscreen agents, insect repellents, vitamins, herbal extracts, antiperspirant and deodorant agents, skin or hair bleaching or coloring agents, depilating agents, anti-fungal and antimicrobial agents, anti-dandruff and anti-acne agents, astringents, and combinations thereof.

Neutralizers useful in compositions of the invention include any pH adjusters listed under CTFA International Cosmetic Ingredients Dictionary and Handbook such as, for example, triethanolamine, amino methyl propanil, sodium hydroxide, ammonium hydroxide, potassium hydroxide, arginine, tetrahydroxypropyl ethylenediamine, PEG-15 cocamine, diisopropanol amine, triisopropanol amine, and combinations thereof.

Humectants useful in compositions of the invention include any humectants listed under CTFA International Cosmetic Ingredients Dictionary and Handbook such as, for example, glycerin, sorbitol, glycol, hydrolyzed wheat protein, polyethylene glycols (PEG), including PEG-4 to PEG-800, and PEG esters, and polyglyceryl sorbitol.

Conditioners useful in compositions of the invention include any hair conditioning agent listed under CTFA International Cosmetic Ingredients Dictionary and Handbook, such as, for example, cationic conditioning quats, mono/di-alkyl quat (such as cetrimonium chloride, stearalkonium chloride, quaternium 82, benenyl trimethyl ammonium chloride, distearyl dimethyl ammonium chloride, hydrogenated tallow alkyl trimethyl ammonium chloride, dialkyl dimethyl ammonium chloride, ditallow alkyl dimethyl ammonium chloride, dicetyl dimethyl ammonium chloride, dibehenyl dimethyl ammonium chloride, stearyl dimethyl benzyl ammonium chloride, stearyl amidopropyl dimethyl ammonium benzyl chloride,), polymeric quats (such as PQ4, PQ-11, guar hydroxypropyltrimethylammonium chloride, PQ-43,44,52,53,55,55,56), hydrolyzed wheat/soy/silk protein, elastin amino acids, lanolin alcohol, PEG40 hydrogenated lanolin, and panthenol.

Conditioners useful in composition of the invention include any hair conditioning agent listed under CTFA International Cosmetic Ingredients Dictionary and Handbook such as, for example, cationic conditioning quats, mono/di-alkyl quat (such as cetrimonium chloride, stearalkonium chloride, quaternium 82, benenyl trimethyl ammonium chloride, distearyl dimethyl ammonium chloride, hydrogenated tallow alkyl trimethyl ammonium chloride, dialkyl dimethyl ammonium chloride, ditallow alkyl dimethyl ammonium chloride, dicetyl dimethyl ammonium chloride, dibehenyl dimethyl ammonium chloride, stearyl dimethyl benzyl ammonium chloride, stearyl amidopropyl dimethyl ammonium benzyl chloride,), polymeric quats (such as PQ-4, PQ-11, guar hydroxypropyltrimethylammonium chloride, PQ-43,44,52,53,55,55,56), hydrolyzed wheat/soy/silk protein, elastin amino acids, lanolin alcohol, PEG-40 hydrogenated lanolin and panthenol.

Silicones useful in compositions of the invention include volatile and non-volatile silicone conditioning agents, such as polydimethylsiloxane (known as dimethicone™), polydimethylsiloxane, polymethylphenylsiloxane, commercially available from GE Silicone, and Dow Corning; polyorganosiloxane materials, polyalkyleneoxide-modified siloxanes, amodimethicone amino-substituted siloxanes, and highly cross-linked polymeric siloxane systems, such as GE SS4230™ and SS4267™, commercially available dimethiconolstm and cyclomethicone™ (Dow Corning 1401™, 1402™, and 1403™ fluids), polymethylphenyl silicones, alkylated silicones, such as methyldecyl silicone and methyloctyl silicone, alkyl-modified siloxanes such as alkyl methicones and alkyl dimethicones with alkyl chains of C10-C50.

Cationic surfactants are also usefully employed as additives in compositions of the invention. Cationic surfactants include, for example, octylbenzyltrimethylammonium chloride, hexadecyltrimethylammonium chloride, hexadecyltrimethylammonium bromide and dodecyltrimethylammonium chloride), oxygen containing amines, quaternary amine salts, ester groups containing quaternary ammonium salts, as disclosed in European patent Publication Nos. EP 0 345 842A2, EP 0 239 910 and U.S. Pat. No. 4,137,180, methylbis (tallowamidoethyl)(2-hydroxyethyl) ammonium methyl sulphate, methyl bis (hydrogenated tallowamido ethyl)(2 hydroxyethyl) ammonium methyl sulphate, imidazolinium salts, including 1-methyl-1-(tallowylamido) ethyl-2-tallowyl-4,5 dihydro imidazolinium methosulphate, and 1-methyl-1-(palmitoylamido) ethyl-2-octadecyl-4,5-dihydroimidazolinium chloride.

Non-ionic surfactants are also usefully employed as additives in compositions of the invention. Typical nonionic surfactants useful in the present invention include, for example, (C₆-C₁₈)alkylphenol alkoxylates (such as t-octyl phenol and nonylphenol ethoxylates having 1-70, and preferably 5-16, ethyleneoxide units), (C₁₂-C₂₀)alkanol alkoxylates and block copolymers of ethylene oxide and propylene oxide. Optionally, the end groups of polyalkylene oxides can be blocked, whereby the free OH groups of the polyalkylene oxides can be etherified, esterified, acetalized and/or aminated. Another modification consists of reacting the free OH groups of polyalkylene oxides with isocyanates. Useful non-ionic surfactants also include, for example, (C₄-C₁₈)alkyl glucosides as well as the alkoxylated products obtainable therefrom by alkoxylation, particularly those obtainable by reaction of alkyl glucosides with ethylene oxide.

Amphoteric or zwitterionic surfactants (such as cocamidopropyl betaine) including both acidic and basic hydrophilic groups are also useful as additives in compositions of the present invention.

Anionic surfactants may also be used as additives. Typical anionic surfactants include, for example, (C₈-C₁₈)alkyl carboxylic acids, (C₁₂-C₂₀)sulfonic acids (sulfonated alkylaryl compounds such as sodium dodecylbenzenesulfonate), (C₁₀-C₂₀)sulfuric acid esters (sulfated alcohols such as lauryl and cetyl sulfates, sodium salts), phosphate esters and salts thereof.

Examples of such surfactants include, for example, any surfactants listed under CTFA International Cosmetic Ingredients Dictionary and Handbook including, for example, PPG-5-ceteth-20 (Proetyl AWS™, available from Croda), PEG-40 hydrogenated castor oil (Tagat™ CH 40, available from Goldschmidt), Oleth-20 (Brij 98™ available from Uniqema), Isoceteth-20 (Arlasolve™ 200 available from Uniqema), Nonoxynol-10 (Makon™ 10 available from Stepan), Polysorbate-20 (Tween™ 20 available from Uniqema).

A typical hair styling gel will have a composition similar to the following:

| Ingredient | Wt. % |
|---|---|
| D.I. water | q.s to 100 |
| Alcohol | 0-90% A.I. |
| Rheology Modifier | 0.1-15% A.I. |
| Hair Fixative Polymer | 0-15% A.I. |
| Neutralizer | 0-15% A.I. |
| Humectant | 0-15% A.I. |
| Surfactant | 0-15% A.I. |
| Conditioning Agent | 0-15% A.I. |
| Silicone | 0-15% A.I. |
| Color | 0-15% A.I. |
| Fragrance | 0-15% A.I. |
| Fragrance Solubilizer | 0-10% AI |
| Preservatives | 0-15% A.I. |

### EXAMPLES

In the following examples, all ratios, parts and percentages are expressed by weight unless otherwise specified, and all reagents used are of good commercial quality unless otherwise specified. The following abbreviations are used in the Examples:

Brookfield viscosities of compositions and formulations were measured using a commercially available Brookfield viscometer. Details of the Brookfield viscosity measurements, and interpretation of Brookfield values are described by Christopher W. Macosko in “Rheology: Principles, Measurements and Applications, VCH Publishers: New York, 1994.

Test Methods

1. High Humidity Curl Retention (HHCR)

The curl retention properties of hair styling product/resin were measured at 25±2° C./90±2% RH over a period of time (up to 4 hr). The less change in percentage curl retention vs. time is an indication of longer lasting hold performance of a hair styling product/resin.

Hair swatches were purchased from International Hair Importer, cut to 3.5 g and 8″ (referred as initial L₀). The hair swatches were then washed by 10% stripping shampoo solution, comb through during rinsing with luke warm water. Excess water was squeezed out, then 0.5 g of sample product was applied to hair using a 1 cc syringe. Curled hair on a ⅝″ curler, secure with metal hair pin. Dried hair on the curler in 45° C. oven for 1 hr, then continued drying at room temperature for 12 hour on a horizontal surface. To start the test, hair clips were removed, and hair slid off the curler. Hair curls were suspended on a plexiglass board. Initial reading of the curl heights (Lᵢ) were measured. Curled hair samples were placed on a board located in a humidity and temperature control chamber. Curl heights were measured at specific time intervals (Lₜ). Percentage (%) curl retention is calculated as L₀−L_{f}/L₀−Lᵢ*100

2. Curl Stiffness and Stiffness Retention

Curl stiffness and stiffness retention properties were measured at 77±2F/50±2% RH. Good curl stiffness is an indication of crunchy curl and stiff hold, high stiffness retention values indicate durable hold.

Hair swatches were prepared in the same manor as in HHCR test. The hair swatches were then tested using a Diastron MTT160 Miniature Tensile Tester-Cyclic Test Method.

The hair swatches were compressed at 60 mm/min, up to 66% compression for 5 cycles. The initial reading of the force of resistance at 1^{st} compressing cycle is recorded as Curl Stiffness.

The percent difference of resistance force between the 1^{st} and 5^{th} compressing cycle is calculated as Stiffness Retention.

### Example 1

A hair styling composition was prepared as follows:

Composition:

| Trade Name | CTFA/INCI Name | Supplier | % Wt. |
|---|---|---|---|
| Water | Water | | 93.28 |
| Aculyn ™ 28 rheology | Acrylates/Beheneth-25 | Rohm and | 4.00 |
| modifier (20% Solids) | Methacrylate Copolymer | Haas | |
| PVP K-90 | Polyvinylpyrrolidone | ISP | 2.00 |
| AMP-95 ™ surfactant | Aminomethyl propanol | Angus | 0.32 |
| Methylparaben NF | Methylparaben | RITA | 0.20 |
| Propylparaben NF | Propylparaben | RITA | 0.10 |
| Neolone ™ 950 | Methylisothiazolinone | Rohm and | 0.10 |
| preservative (0.95% A.I.) | | Haas | |

The composition was prepared as follows: the rheology modifier (Acrylates/Beheneth-25 Methacrylate Copolymer) was blended with one half of the water; the hair styling resin (PVP K-90) was blended with the remaining water along with the other components of the composition; the two blends were added together and mixed until a clear viscous gel formed. The gel had the following properties:

| Parameter | Result | Method |
|---|---|---|
| Viscosity* | 22,000 cps | Brookfield RV viscometer, |
| | | Spindle #6, 20 rpm |
| PH | 6.06 | pH meter |
| Appearance | Clear | Visual |

### Comparative Examples 1 and 2

Comparative Example 1 was prepared in a manner similar to Example 1 except that the rheology modifier used was Carbopol™ 940 rheology modifier. Comparative Example 2 was a commercially available hair gel wherein the rheology modifier is Carbopol and the hair styling resin is PVP.

Example 1, Comparative Example 1 and Comparative Example 2 were evaluated for high humidity curl retention and curl stiffness and retention using the procedures described above. The results of the evaluations were as follows:

High Humidity Curl Retention Performance (90% RH, 23° C.)

| Sample | T = 0 | T = 1 hr | T = 2 hr | T = 4 hr |
|---|---|---|---|---|
| Example 1 | 100% | 99.6% | 99.6% | 99.6% |
| Comp. Example 1 | 100% | 89.2% | 70.0% | 69.6% |
| Comp. Example 2 | 100% | 15.3% | 15.3% | 15.3% |

Curl Stiffness and Stiffness Retention by Dia-Stron™ Curl Compression Test

| | Initial | Stiffness Retention after |
|---|---|---|
| Sample | Stiffness(gmf) | 5^{th} Compression (%) |
| Example 1 | 411.5 | 74 |
| Comp. Example 1 | 283 | 78 |

In addition, the resistance to sheer thinning of Examples 1 and 3 were evaluated with the following results:

Rheology Profiles of Styling Gels

| | Comparative Example 2 | Example 1 |
|---|---|---|
| Speed | Viscosity (cps) | Viscosity (cps) |
| 6.00 | 25466.67 | 51500 |
| 12.00 | 13900.00 | 31833 |
| 20.00 | 9040.00 | 22000 |
| 30.00 | 6480.00 | 16300 |

These data show the advantages provided to hair styling compositions incorporating the combinations of rheology modifiers and hair fixative resins of this invention.

We claim:

1. A hair styling composition comprising:
a) one or more rheology modifiers selected from the group consisting of non-crosslinked hydrophobically modified poly(meth)acrylates, amino modified acrylates, acrylonitrogen modified (meth)acrylates, hydrophobically modified polyethers, and combinations thereof,
b) one or more hair fixative resins selected from the group consisting of: acrylates/hydroxyestersacrylates copolymer, AMP-acrylates/allyl methacrylate copolymer, butyl ester of PVM/MA copolymers, ethyl ester of polyvinyl methyl ether/methacrylate copolymers, ethyl ester of PVM/MA copolymer, guar hydroxypropyl trimonium chloride, guar hydroxypropyl trimonium chloride, isobutylene/ethylmaleimide/hydroxyethylmaleimide copolymers, modified corn starch, octylacrylamide/acrylates butylaminoethyl methacrylate copolymer, octylacrylamide/acrylates butylaminoethyl methacrylate copolymers, poly(methacrylic acid/acrylamidomethyl propane sulfonic acid), Polyimide-1, polyquaternium-10, polyquaternium-11, polyquatemium-16, polyquatemium-2, polyquatemium-28, polyquatemium-28 and dimethicones, polyquatemium-39, polyquatemium-4, polyquatemium-46, polyquaternium-55, polyquaternium-7, polyvinylcaprolactam acrylate copolymers, polyvinylpyrrolidinone, PVP and dimethicones, PVP/dimethylaminoethyl methacrylate copolymers, PVP/dimethylaminoethylmethacrylate copolymer, PVP/DMAPA acrylate copolymers, PVP/DMAPA acrylates copolymer, PVP/vinyl acetate copolymer, PVP/vinylcaprolactam/DMAPA acrylate copolymers, VA/Crotonate copolymer, VA/Crotonates/vinyl neodecanoate copolymer, vinyl acetate/crotonates/vinyl neodecanoate copolymers, vinyl acetate/crotonic acid copolmer, vinyl acetate/crotonic acid copolymers, vinyl caprolactam/PVP/dimethyl aminoethyl methacrylate copolymer, vinyl caprolactam/PVP/dimethyl aminoethyl methacrylate copolymers, VP/acrylates/lauryl methacrylate copolymer, and combinations thereof.

2. The hair styling composition of claim 1 further comprising one or more additives.

3. The hair styling composition of claim 2 wherein the one or more additives is selected from thickeners, additional rheology modifiers, additional hair fixative resins, other polymers, neutralizers, humectants, surfactants, conditioning agents, silicones, colors, dyes, fragrances, fragrance solubilizers, naturally occurring materials, and preservatives.

4. A process for treating and modifying hair comprising the step of applying to hair a hair styling composition comprising:
a) one or more rheology modifiers selected from the group consisting of non-crosslinked hydrophobically modified poly(meth)acrylates, amino modified acrylates, acrylonitrogen modified (meth)acrylates, hydrophobically modified polyethers, and combinations thereof,
b) one or more hair fixative resins selected from the group consisting of: acrylates/hydroxyesters acrylates copolymer, AMP-acrylates/allyl methacrylate copolymer, butyl ester of PVM/MA copolymer, butyl ester of PVM/MA copolymers, ethyl ester of polyvinyl methyl ether/methacrylate copolymers, ethyl ester of PVM/MA copolymer, guar hydroxypropyl trimonium chloride, guar hydroxypropyl trimonium chloride, isobutylene/ethylmaleimide/hydroxyethylmaleimide copolymers, modified corn starch, octylacrylamide/acrylates butylaminoethyl methacrylate copolymer, octylacrylamide/acrylates butylaminoethyl methacrylate copolymers, poly(methacrylic acid/acrylamidomethyl propane sulfonic acid), Polyimide-1, polyquaternium-10, polyquaternium-11, polyquatemium-16, polyquaternium-2, polyquatemium-28, polyquaternium-28 and dimethicones, polyquatemium-39, polyquaternium-4, polyquatemium-46, polyquaternium-55, polyquaternium-7, polyvinylcaprolactam acrylate copolymers, polyvinylpyrrolidinone, PVP and dimethicones, PVP/dimethylaminoethyl methacrylate copolymers, PVP/dimethylaminoethylmethacrylate copolymer, PVP/DMAPA acrylate copolymers, PVP/DMAPA acrylates copolymer, PVP/vinyl acetate copolymer, PVP/vinylcaprolactam/DMAPA acrylate copolymers, VA/Crotonate copolymer, VA/Crotonates/vinyl neodecanoate copolymer, vinyl acetate/crotonates/vinyl neodecanoate copolymers, vinyl acetate/crotonic acid copolmer, vinyl acetate/crotonic acid copolymers, vinyl caprolactam/PVP/dimethyl aminoethyl methacrylate copolymer, vinyl caprolactam/PVP/dimethyl aminoethyl methacrylate copolymers, VP/acrylates/lauryl methacrylate copolymer, and combinations thereof; and
optionally, c) one or more additives.

5. A hair styling composition comprising:
a) a rheology modifier;
b) a hair fixative resin; and
optionally c) one or more additives;
wherein both the rheology modifier and the hair fixative resin consist of acrylates beheneth-25 methacrylate copolymer.
